(19) United States
(12) Patent Application Publication
(10) Pub. No.: US 2005/0103807 A1
(43) Pub. Date: May 19, 2005

(54) Dispenser assembly

(76) Inventor: Patrick Murphy, Springfield, PA (US)

Correspondence Address: Eugene E. Renz, Jr., P.C., 205 North Monroe Street, Post Office Box 2056, Media, PA, 19063-9056, US

(21) Appl. No.: 10/989,987

(22) Filed: Nov. 16, 2004

Related U.S. Application Data
(60) Provisional application No. 60/520,631, filed on Nov. 17, 2003.

(51) Int. Cl. A61K 7/32; G01F 11/00

(52) U.S. Cl. 222/226

(57) ABSTRACT

A method of forming a solid substance from a fluid material in a dispenser having a container and a piston in the container actuatable longitudinally therein to selectively displace the solid substance in the container and comprising the steps of positioning the piston at its lower most limit position placing an elongated hollow rod over the threaded shaft of the piston actuating mechanism so that one axial end projects beyond the discharge end of the container filling the container with a liquid substance up to the discharge end withdrawing the rod from the dispenser when the substance has hardened thereby forming a longitudinal chamber in the solid substance spaced radially from the threaded actuator shaft. A dispenser assembly comprising a container a cap which fits over the open end of the container a piston mounted in the container having a cross section conforming to the cross section of the container and having a depending internally threaded collar an actuator mechanism including a handwheel and an elongated externally threaded shaft which engages and mates with the collar in the piston a solid substance to be dispensed of a cross section generally similar to the cross section of the container mounted on the piston and having a longitudinally extended opening circumscribing the threaded shaft of said actuator and radially spaced therefrom.

### CROSS REFERENCE TO RELATED APPLICATIONS

This application claims the benefit of U.S. Provisional Application No. 60/520,631 filed Nov. 17, 2003, and entitled, SURF WAX DISPENSER ASSEMBLY.

### FIELD OF THE INVENTION

The present invention relates to dispenser assemblies and more specifically to improvements in dispensers used for substances such as wax products for application, for example, to snowboards, skateboards, surfboards and skis. The present invention also relates to a method for preparing and filling the dispenser with wax in a manner to insure trouble-free operation of the mechanism for displacing the wax for application to snowboards, skateboards, surfboards and skis.

### SUMMARY OF THE INVENTION

To this end, the dispenser comprises a container and a cap, a piston mounted in the container of a cross section complementing the cross section of the container, a wax block supported on the piston and a threaded shaft rotatable by a handwheel to advance the wax beyond the discharge end of the container for application to a ski or surfboard. After use, the handwheel can be rotated in the opposite direction to recess the wax into the container.

The wax block can be formed in situ in the container by simply placing an elongated hollow road over the threaded actuator shaft and then depositing liquid wax to form the block. When the wax hardens, the rod is withdrawn creating an elongated chamber for the actuator shaft and a radial clearance or space between the wax bore and shaft to insure easy and free rotation of the shaft.

With the above in mind, it is an object of the present invention to provide a container-closure assembly for dispensing wax which is characterized by novel features of construction and arrangement facilitating forming the wax in the container in a predetermined manner insuring free and easy operation of the mechanism for advancing the wax in the container as it is used.

Another object of the present invention is to provide a dispenser which is economical to fill with the wax product and which permits the user to dispense preselected quantities on the surface of skis and surfboards for example in a quick and easy fashion.

### BRIEF DESCRIPTION OF THE DRAWINGS

These and other objects of the present invention and various features and details of the operation and construction thereof are herein after more fully set forth with reference to the accompanying drawings wherein:

FIG. 1A. is an exploded side elevational view showing the components comprising the DISPENSER ASSEMBLY of the present invention;

FIG. 1B. is a side elevational view of a DISPENSER ASSEMBLY in accordance with the present invention;

FIG. 2A. is a bottom plan view of the cap;

FIG. 2B. is a sectional view of the cap taken on lines 2B-2B of FIG. 2a;

FIG. 3A. is a top plan view of the piston;

FIG. 3B. is a sectional view of the piston taken on lines 3B-3B of FIG. 3A;

FIG. 3C. is a bottom plan view of the piston;

FIG. 4A. is a top plan view looking down into the container;

FIG. 4B. is a transverse sectional view of the container taken on lines 4B-4B of FIG. 4A;

FIG. 4C. is a bottom plan view of the container;

FIG. 5A. is a transverse sectional view through the DISPENSER ASSEMBLY with all the components including the cap and actuator mechanism in place;

FIG. 5B. is an enlarged sectional view of the portion circled in FIG. 5A showing a portion of the actuator mechanism;

FIG. 6. is a transverse sectional view with the rod in place as a first step in forming the wax product to be dispensed;

FIG. 7. is a similar sectional view showing the next step namely introduction of liquid wax; and

FIG. 8. shows the complete assembly with the rod removed showing the gap between the threaded actuator shaft and the hardened wax block.

### DESCRIPTION OF PREFERRED EMBODIMENT

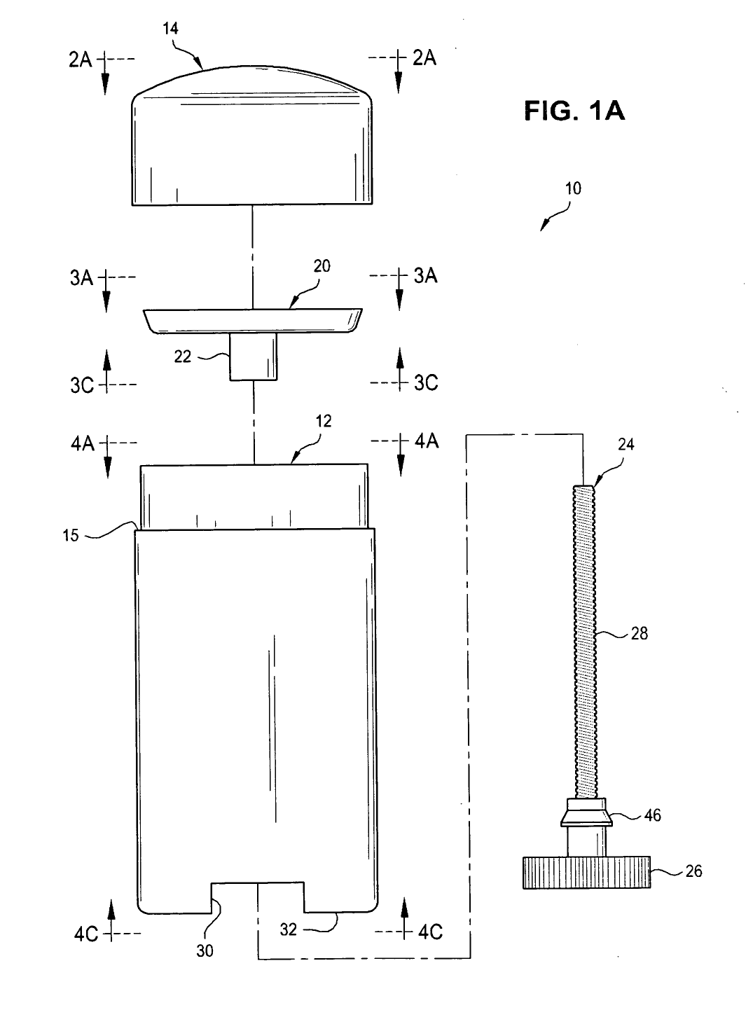

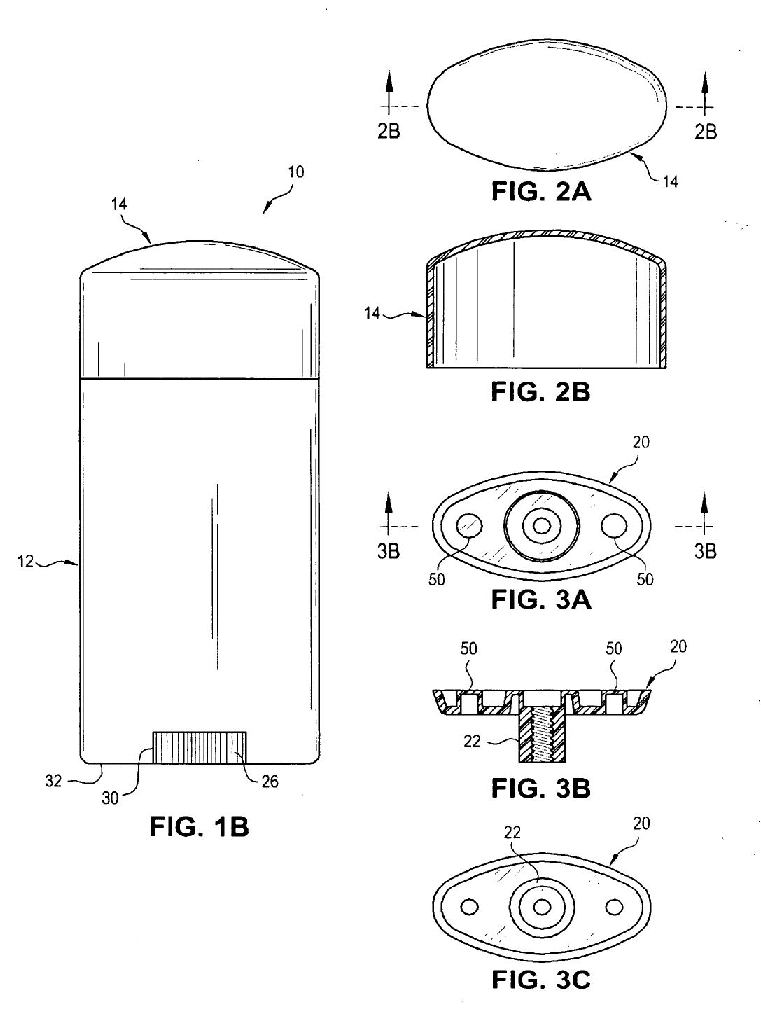

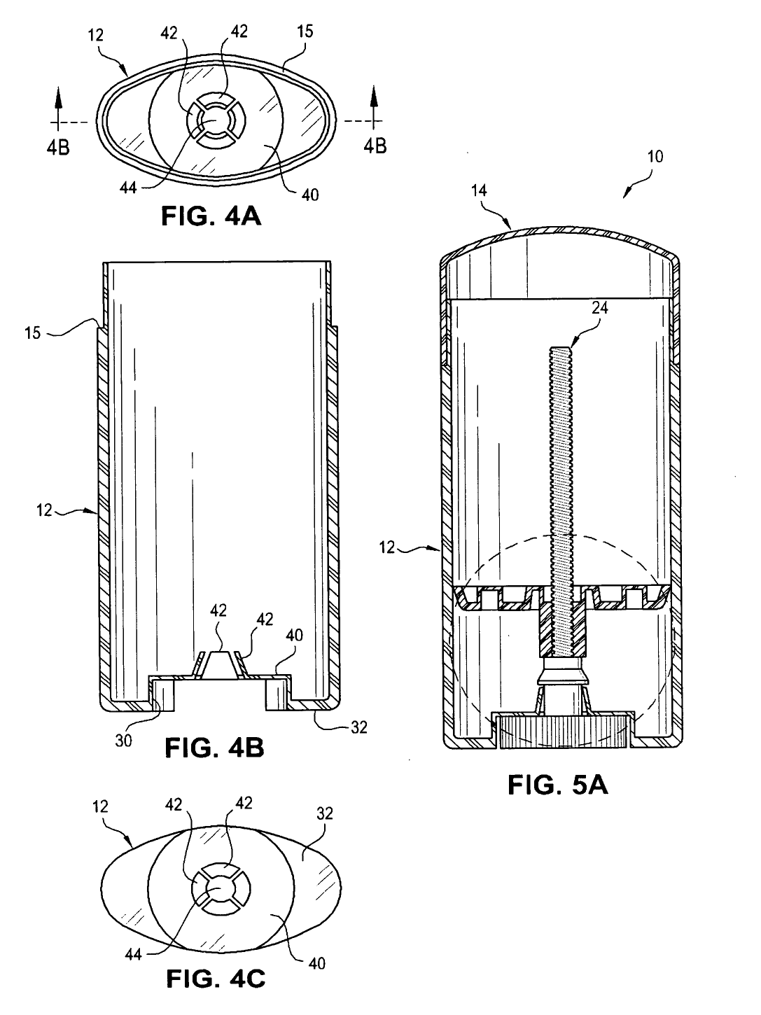

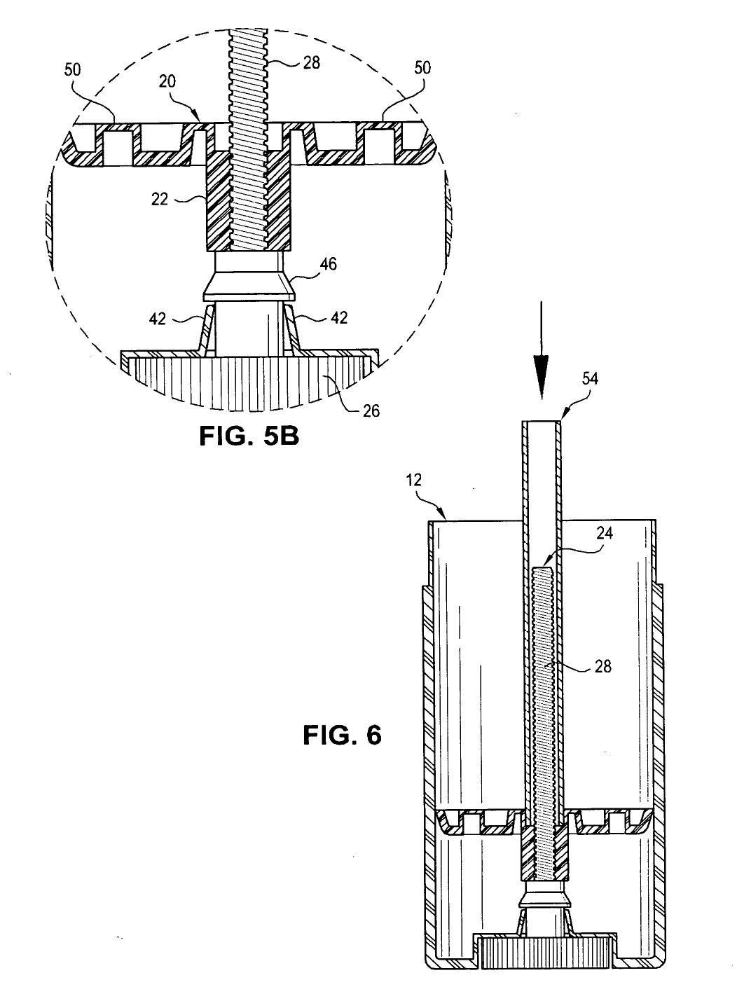

Referring now to the drawings and to particularly to FIGS. 1A, 1B and 8 thereof, there is shown a wax dispenser assembly in accordance with the present invention generally designated by the numeral 10. The open discharge end of the container (12) is stepped to provide a seat (15) for the cap (14) when it is in place over the open end of the container. A wax block W having a cross section conforming general to the cross section of the container (12) sits on a piston (20) which has a depending internally threaded collar (22). An actuator mechanism generally designated by the numeral (24) is operatively connected to the piston (20) so that the piston (20) may be selectively actuated axially in the container (12) to displace the wax block W forwardly so that its upper axial end clears the opening in the upper end of the container thereby permitting a user to apply wax to a curved or flat surface such as a snowboard or surfboard.

The actuator mechanism (24) includes a handwheel (26) having an elongated threaded shaft (28) which engages through the collar (22) so that rotation of the handwheel (26) in one direction produces an axial movement of the piston (20) and rotation of the handwheel (26) in the opposite direction retracts the piston (20). The handwheel nests in a cutout (30) in the base (32) which exposes a portion of the handwheel on opposing sides so that a user can rotate the shaft (28) to displace the piston (20) in the manner described and yet the handwheel is flush with the base (32) of the container (12) so that it can be placed on a planar surface in an upright position when not in use. The base (32) of the container has an enlarged generally circular shaped opening (40) having a series of circumferentially spaced flexible tabs (42) which taper upwardly to define at their outer edges a generally circular opening (44). The shaft has a frusto conical projection (46) at its lower end adjacent the handwheel (26) and spaced slightly upwardly therefrom which on assembly of the shaft through the opening (44), presses the tabs (42) radially outwardly so that when the shaft (28) is fully seated the lower end of the frusto conical projection (40) engages the outer free ends of the tabs (42) to lock the shaft in the position indicated permitting rotation of the shaft to achieve the wax block displacement action described above.

The piston (20) has two axially extending lugs (50) on either side of the collar (22) which press the lower face of the wax block to aid in retaining the wax block Bw on the upper face of the piston.

The wax block Bw has an elongated longitudinally extending channel (52) of a diameter D greater than the diameter Ds of the threaded portion of the shaft (28) to facilitate axial displacement of the piston without gumming up the threads of the shaft which could adversely interfere with rotation of the shaft.

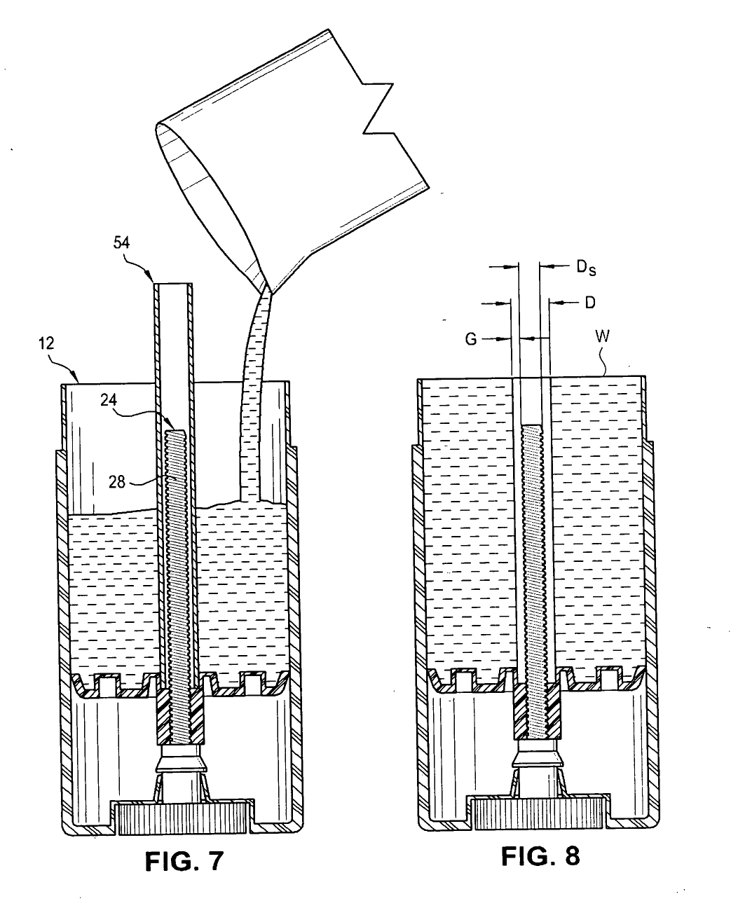

The dispenser assembly is of a design and configuration so that the wax block Bw can be formed in situ thereby minimizing assembly time and expense compared to separate molding of the wax block Bw. Accordingly, the handwheel (26) turns so that the piston is in its lower most position shown in FIG. 6. With the cap removed, an elongated hollow tubular rod (54) engages over the threaded shaft Bw and is of a length Li so that the tip end (56) protrudes sufficiently beyond the block so that it can be gripped by a user perhaps an inch to an inch and a half beyond the upper discharge end of the container (See FIG. 6). Liquid wax is discharged into the container to a level adjacent the top edge of the container. After the wax is cooled and solidified, the rod (54) is simply withdrawn to provide a circumferential gap G between the threaded shaft (28) and the longitudinal channel (52) formed in the wax block Bw a sufficient space so that activation of the shaft does foul the shaft threads.

Even though a particular embodiment of the invention has been illustrated and described herein, it is not intended to limit the invention and changes and modifications may be made therein within the scope of the following claims.

1. A method of forming a solid substance from a fluid material in a dispenser having a container and a piston in the container actuatable longitudinally therein to selectively displace the solid substance in the container and comprising the steps of:
positioning the piston at its lower most limit position;
placing an elongated hollow rod over the threaded shaft of the piston actuating mechanism so that one axial end projects beyond the discharge end of the container;
filling the container with a liquid substance up to the discharge end;
withdrawing the rod from the dispenser when the substance has hardened thereby forming a longitudinal chamber in the solid substance spaced radially from the threaded actuator shaft.

2. A dispenser assembly comprising a container;
a cap which fits over the open end of the container;
a piston mounted in the container having a cross section conforming to the cross section of the container and having a depending internally threaded collar;
an actuator mechanism including a handwheel and an elongated externally threaded shaft which engages and mates with the collar in the piston;
a solid substance to be dispensed of a cross section generally similar to the cross section of the container mounted on the piston and having a longitudinally extended opening circumscribing the threaded shaft of said actuator and radially spaced therefrom.
